(19) United States
(12) Patent Application Publication
(10) Pub. No.: US 2007/0027107 A1
(43) Pub. Date: Feb. 1, 2007

(54) Compositions and methods for treating estrogen-dependent diseases and conditions

(76) Inventor: Curt Hendrix, Encino, CA (US)

Correspondence Address: CATALYST LAW GROUP, APC, 9710 SCRANTON ROAD, SUITE S-170, SAN DIEGO, CA, 92121, US

(21) Appl. No.: 11/495,284

(22) Filed: Jul. 28, 2006

Related U.S. Application Data
(60) Provisional application No. 60/703,730, filed on Jul. 29, 2005.
(60) Provisional application No. 60/733,541, filed on Nov. 3, 2005.

(51) Int. Cl. A61K 31/737; A61K 31/729

(52) U.S. Cl. 514/54

(57) ABSTRACT

A pharmaceutical composition for the treatment of an estrogen-dependent disease or condition comprises: (1) at least one polysaccharide selected from the group consisting of an alginate and a fucoidan in a quantity effective to treat an estrogen-dependent disease or condition; and (2) a pharmaceutically acceptable carrier. The composition can include both an alginate and a fucoidan. The composition can include other ingredients such as at least one compound selected from the group consisting of diindolylmethane and indole-3-carbinol in a quantity sufficient to inhibit the activity of estrogen. Methods for use of the composition for the treatment of an estrogen-dependent disease or condition, especially endometriosis, are described.

### CROSS-REFERENCES

This application claims priority from U.S. Provisional Application Ser. No. 60/733,541, entitled “Compositions and Methods for Treating Estrogen-Dependent Diseases and Conditions,” by Curt Hendrix, filed on Nov. 3, 2005, which provisional application is incorporated herein by this reference. This application also claims priority from U.S. Provisional Application Ser. No. 60/703,730, entitled “Use of Seaweed Extracts to Treat or Prevent Estrogen-Dependent Diseases,” by Curt Hendrix, filed on Jul. 29, 2005, which provisional application is also incorporated herein by this reference.

### BACKGROUND OF THE INVENTION

This invention is directed to compositions and methods for treating estrogen-dependent diseases and conditions, most particularly in females, especially endometriosis.

Endometriosis is an important and widely-occurring clinical problem in women. The exact etiology of endometriosis is not known. Endometriosis is frequently associated with dysmenorrhea, dyspareunia, chromic pain, and infertility. Endometriosis is an estrogen dependent disease characterized by the growth of endometrial stromal cells and glands outside of the uterus.

Endometriosis is a chronic disease that is associated with significant morbidity and is a leading cause of hospitalization for gynecologic surgery. Between 6 and 15% of women at all reproductive ages have been diagnosed with endometriosis. Significant pain on menstruation and intercourse, leading to health distress and interference with normal activities such as work and leisure time activities are common problems in women with endometriosis. In addition, endometriosis is a major cause of infertility. A recent study has estimated that medical costs associated with endometriosis in the United States alone are approximately $3.6 billion annually. Taken together, endometriosis represents a significant burden on the health care system and has a considerable impact on the quality of life for women with endometriosis. Current methods for the medical management of endometriosis are associated with treatment failures and undesirable side effects that limit their use. Therefore, novel therapeutic strategies that effectively ameliorate the growth of endometriotic implants and preserve fertility are needed.

In the etiology of endometriosis, endometrial cells may be carried up through the uterus into the pelvis during menstruation or they may travel to other parts of the body via the circulatory system. Endometriosis is a chronic and usually progressive condition.

Women with endometriosis and their families are at a higher risk of developing autoimmune diseases, such as diabetes and thyroid disorders, and some cancers, such as breast and ovarian cancer, melanoma (an aggressive form of skin cancer) and non-Hodgkin's lymphoma. In women with endometriosis, for example, the risk of developing diabetes is as high as 42%. The risk in the general population is 5.9%. The incidence of hypothyroidism (underactive thyroid) also is higher: 6.8% in women with endometriosis compared to 1.9% in the general population.

Women with endometriosis and their families are at higher risk of developing breast cancer (26.9% compared to 0.1% in the general population), melanoma (9.8% compared to 0.01%) and ovarian cancer (8.5% compared to 0.04%). The incidence of non-Hodgkin's lymphoma also is higher in women with endometriosis. Endometriosis is a disease affecting an estimated 77 million women and teens worldwide. It is a leading cause of infertility, chronic pelvic pain and hysterectomy.

Findings of one of the largest surveys conducted of over 4,000 Endometriosis patients in the United States and Canada have indicated possible links to other serious medical conditions, including a 9.8% incidence of melanoma, compared with 0.01% in the general population, a 26.9% incidence of breast cancer, compared with 0.1% in the general population; and an 8.5% incidence of ovarian cancer, compared with 0.04% in the general population. Women with endometriosis who participated in the survey also had a greater incidence of autoimmune conditions and Meniere's disease. There is also evidence linking endometriosis with autoimmune disorders, endocrine disorders, fibromyalgia, chronic fatigue syndrome, and atopic diseases (N. Sinaii et al., “High Rates of Autoimmune and Endocrine Disorders, Fibromyalgia, Chronic Fatigue Syndrome and Atopic Diseases Among Women with Endometriosis: A Survey Analysis,” Hum. Reprod. 17: 2715-2724 (2002)), incorporated herein by this reference.

Epidemiological studies show that incidence rates of estrogen-dependent diseases such as cancers of the breast, endometrium and ovary are among the highest in Western, industrialized countries, while rates are much lower in China and Japan (D. M. Parkin et al., “Estimates of the Worldwide Incidence of 25 Major Cancers in 1990,” Int. J. Cancer 80: 827-841 (1999); D. M. Parkin et al., “Global Cancer Statistics,” CA Cancer J. Clin. 49: 33-64 (1999)). These disparities can be attributable, in part, to differences in dietary and environmental exposures associated with affluent and modern lifestyles that promote estrogenic stimulation and hormone imbalances (M. P. Madigan et al., “Serum Hormone Levels in Relation to Reproductive and Lifestyle Factors in Postmenopausal Women (United States), Cancer Causes Control 9: 199-207 (1998); A. McTiernan, “Behavioral Risk Factors in Breast Cancer: Can Risk Be Modified,” Oncologist 8: 326-334 (2003); A. Tavani et al., “Influence of Selected Hormonal and Lifestyle Factors on Familial Propensity to Ovarian Cancer,” Gynecol. Oncol. 92: 922-926 (2004)). Although the mechanisms are not fully understood, epidemiological and experimental data suggest that exposure to estrogens, through endogenous production and exogenous exposures resulting in an imbalance in the estrogen/progesterone ratio, can be the most critical determinants in disease risk (J. A. Cauley et al., “Elevated Serum Estradiol and Testosterone Concentrations Are Associated With a High Risk for Breast Cancer. Study of Osteoporotic Fractures Research Group,” Ann. Int. Med. 130: 270-277 (1999); S. E. Hankinson et al., “Plasma Sex Steroid Hormone Levels and Risk of Breast Cancer in Postmenopausal Women,” J. Natl. Cancer Inst. 90: 1292-1299 (1998); G. B. Baskin et al., “Endometrial Hyperplasia, Polyps, and Adenomyosis Associated with Unopposed Estrogen in Rhesus Monkeys (Macaca mulatta),” Vet. Pathol. 39: 572-575 (2002)). In estrogen-sensitive tissues, estrogen triggers cell proliferation, and through prolonged stimulation, hyperplasia (W. Yue et al., “Genotoxic Metabolites of Estradiol in Breast: Potential Mechanism of Estradiol Induced Carcinogenesis,” J. Steroid Biochem. Mol. Biol. 86: 477-486 (2003)) and possibly neoplasia can occur. Reproductive factors associated with increased exposure to menstruation resulting in persistent and sustained estrogenic stimulation, such as shorter menstrual cycles, reduced parity, early menarche, and late menopause, are known to increase risk of endometriosis and estrogen-dependent cancers (M. Hinkula et al., “Grand Multiparity and Incidence of Endometrial Cancer: A Population-Based Study in Finland,” Int. J. Cancer 98: 912-915 (2002); M. Daniels et al., “Associations Between Breast Cancer Risk Factors and Religious Practices in Utah,” Prev. Med. 38: 28-38 (2004)), while post-menopausal obesity, hormone replacement therapy and alcohol consumption can be associated with increased breast cancer risk (T. J. Key et al. “Body Mass Index, Serum Sex Hormones, and Breast Cancer Risk in Postmenopausal Women,” J. Natl. Cancer Inst. 95: 1218-1226 (2003); V. Beral, “Breast Cancer and Hormone-Replacement Therapy in the Million Women Study,” Lancet 362: 419-427 (2003); A. Tjonneland et al., “Alcohol Intake, Drinking Patterns and Risk of Postmenopausal Breast Cancer in Denmark: A Prospective Cohort Study,” Cancer Causes Control 14: 277-284 (2003)).

A review of endometriosis treatment methods is found in A. K. Schroder et al., “Medical Management of Endometriosis: a Systematic Review,” Drugs 7: 451-463 (2004), incorporated herein by this reference. Treatment of endometriosis with an aromatase inhibitor is described in E. R. Shippen & W. J. West, Jr., “Successful Treatment of Severe Endometriosis in Two Premenopausal Women with an Aromatase Inhibitor,” Fertil. Ster. 81: 1395-1398 (2004), incorporated herein by this reference. Other treatment modalities include treatment with gonadotropin-releasing hormone antagonists (GnRH-As) or with danazol. However, these treatment methods have high incidences of serious side effects and cannot be used for extended periods of time. They can lead to increased cholesterol levels, bone loss, insomnia, disturbances of sexual functioning, and depression, among other possible side effects.

Therefore, there is a need for improved treatments for endometriosis and other estrogen-dependent conditions and diseases. There is a particular need for improved treatments that are well-tolerated with few side effects and that can be used together with other treatments. There is also a particular need for treatments that prevent the sequelae associated with endometriosis, in particular malignancies.

### SUMMARY OF THE INVENTION

One aspect of the invention is a pharmaceutical composition for the treatment of an estrogen-dependent disease or condition, the composition comprising:

(1) at least one polysaccharide selected from the group consisting of an alginate and a fucoidan in a quantity effective to treat an estrogen-dependent disease or condition; and

(2) a pharmaceutically acceptable carrier.

Typically, the estrogen-dependent disease or condition is endometriosis.

The composition preferably comprises both an alginate and a fucoidan, but can alternatively comprise one of an alginate and a fucoidan. Preferably, the alginate is a polymer of guluronic acid and mannuronic acid.

Typically, the composition comprises a quantity of polysaccharide such that the daily dose of the polysaccharide that is administered is from about 10 mg to about 3000 mg. Preferably, the composition comprises a quantity of polysaccharide such that the daily dose of the polysaccharide that is administered is from about 100 mg to about 1500 mg.

The composition can further include glucaric acid or a salt thereof, such as calcium D-glucarate, in a quantity sufficient to inhibit the enzyme β-glucuronidase. Alternatively, the composition can further include D-glucaro-1,4-lactone in a quantity sufficient to inhibit the enzyme β-glucuronidase.

In another alternative, the composition can further include at least one compound selected from the group consisting of diindolylmethane and indole-3-carbinol in a quantity sufficient to inhibit the activity of estrogen.

In yet another alternative, the composition can further include anastrozole or other aromatase inhibitors in a quantity sufficient to inhibit aromatase.

In still another alternative, the composition can further include progesterone or a progestin in a quantity sufficient to inhibit the effects of estrogen. If the composition includes a progestin, the progestin can be selected from the group consisting of from the group consisting of megestrol acetate, medroxyprogesterone acetate, 19-nortestosterone, norethindrone, ethynodiol diacetate, norgestrel, desogestrel, norgestimate, and their derivatives.

In still another alternative, the composition can further include a lignan or a sterol from an algal source in a quantity sufficient to act as an antagonist for the binding of estrogen to estrogen receptors.

Typically, the composition has the activity of inhibiting the expression of at least one protein selected from the group consisting of aromatase, SF-I, COX-I, COX-II, and 15-hydroxyprostaglandin dehydrogenase at the level of transcription.

Another aspect of the invention is a pharmaceutical composition for the treatment of an estrogen-dependent disease or condition, the composition comprising:

(1) calcium D-glucarate in a quantity sufficient to treat an estrogen-dependent condition;

(2) diindolylmethane in a quantity sufficient to treat an estrogen-dependent condition; and

(3) a pharmaceutically acceptable carrier.

This composition can include additional ingredients such as described above, including, but not limited to, anastrozole, progesterone or a progestin, a lignan from an algal source, or a steroid from an algal source.

Typically, the composition has the activity of inhibiting the expression of at least one protein selected from the group consisting of aromatase, SF-I, COX-I, COX-II, and 15-hydroxyprostaglandin dehydrogenase at the level of transcription.

Another aspect of the invention is a method of treating an estrogen-dependent disease or condition comprising administering an effective quantity of a composition according to the present invention, as described above, to an individual suffering from such a disease or condition, as described above. The estrogen-dependent disease or condition can be endometriosis. The administration of the effective quantity of the composition can inhibit the expression of at least one protein selected from the group consisting of aromatase, SF-I, COX-I, COX-II, and 15-hydroxyprostaglandin dehydrogenase at the level of transcription.

### DETAILED DESCRIPTION OF THE INVENTION

Endometriosis is a disease that affects the physical health and emotional well-being of many women of reproductive age. Endometriosis is defined as the presence of endometrial tissue outside its normal location in the uterus. The disease can range in severity from mild to severe. Patients can be asymptomatic. However, a significant fraction of patients with endometriosis experience symptoms that are severe and potentially incapacitating. These symptoms include dysmenorrheal, dyspareunia, and infertility. The diagnosis of endometriosis can only be confirmed by direct visualization, using techniques such as laparoscopy and biopsy. The risk of endometriosis is increased in women who have an affected first-degree relative, suggesting a genetic component. The risk is also increased in patients who have shorter menstrual cycle length, longer duration of menstrual flow, or who have experienced fewer than two full-term pregnancies. The etiology of endometriosis is not fully understood. Factors contributing to it may include retrograde menstruation, impaired immune function, anatomical abnormalities of the uterus, and genetic factors.

Treatment options for endometriosis are not satisfactory. Options include palliative measures for pain, hormonal therapies to suppress ovarian steroidogenesis and induce endometrial atrophy, and surgery, either to remove visible lesions, or as a last resort, the uterus and ovaries. These treatment options are not satisfactory, because hormone treatment carries with it the risk of serious side effects, and surgical treatment is invasive and carries risks of infection.

Moreover, endometriosis is significant because it is associated with an increased risk of melanoma, breast cancer, ovarian cancer, and autoimmune disease. Therefore, there is a pressing need for more efficient and safe means for the treatment of endometriosis.

Factors related to endometriosis are also believed to be involved in the etiology of other conditions associated with excess estrogen production, including malignancies. Epidemiological studies show that incidence rates of estrogen-dependent diseases such as cancers of the breast, endometrium and ovary are among the highest in Western, industrialized countries, while rates are much lower in China and Japan (D. M. Parkin et al., “Estimates of the Worldwide Incidence of 25 Major Cancers in 1990,” Int. J. Cancer 80: 827-841 (1999); D. M. Parkin et al., “Global Cancer Statistics,” CA Cancer J. Clin. 49: 33-64 (1999)). These disparities can be attributable, in part, to differences in dietary and environmental exposures associated with affluent and modern lifestyles that promote estrogenic stimulation and hormone imbalances (M. P. Madigan et al., “Serum Hormone Levels in Relation to Reproductive and Lifestyle Factors in Postmenopausal Women (United States), Cancer Causes Control 9: 199-207 (1998); A. McTiernan, “Behavioral Risk Factors in Breast Cancer: Can Risk Be Modified,” Oncologist 8: 326-334 (2003); A. Tavani et al., “Influence of Selected Hormonal and Lifestyle Factors on Familial Propensity to Ovarian Cancer,” Gynecol. Oncol. 92: 922-926 (2004)). Although the mechanisms are not fully understood, epidemiological and experimental data suggest that exposure to estrogens, through endogenous production and exogenous exposures resulting in an imbalance in the estrogen/progesterone ratio, can be the most critical determinants in disease risk (J. A. Cauley et al., “Elevated Serum Estradiol and Testosterone Concentrations Are Associated With a High Risk for Breast Cancer. Study of Osteoporotic Fractures Research Group,” Ann. Int. Med. 130: 270-277 (1999); S. E. Hankinson et al., “Plasma Sex Steroid Hormone Levels and Risk of Breast Cancer in Postmenopausal Women,” J. Natl. Cancer Inst. 90: 1292-1299 (1998); G. B. Baskin et al., “Endometrial Hyperplasia, Polyps, and Adenomyosis Associated with Unopposed Estrogen in Rhesus Monkeys (Macaca mulatta),” Vet. Pathol. 39: 572-575 (2002)). In estrogen-sensitive tissues, estrogen triggers cell proliferation, and through prolonged stimulation, hyperplasia (W. Yue et al., “Genotoxic Metabolites of Estradiol in Breast: Potential Mechanism of Estradiol Induced Carcinogenesis,” J. Steroid Biochem. Mol. Biol. 86: 477-486 (2003)) and possibly neoplasia can occur. Reproductive factors associated with increased exposure to menstruation resulting in persistent and sustained estrogenic stimulation, such as shorter menstrual cycles, reduced parity, early menarche, and late menopause, are known to increase risk of endometriosis, as well estrogen-dependent cancers (M. Hinkula et al., “Grand Multiparity and Incidence of Endometrial Cancer: A Population-Based Study in Finland,” Int. J. Cancer 98: 912-915 (2002); M. Daniels et al., “Associations Between Breast Cancer Risk Factors and Religious Practices in Utah,” Prev. Med. 38: 28-38 (2004)), while post-menopausal obesity, hormone replacement therapy and alcohol consumption can be associated with increased breast cancer risk (T. J. Key et al. “Body Mass Index, Serum Sex Hormones, and Breast Cancer Risk in Postmenopausal Women,” J. Natl. Cancer Inst. 95: 1218-1226 (2003); V. Beral, “Breast Cancer and Hormone-Replacement Therapy in the Million Women Study,” Lancet 362: 419-427 (2003); A. Tjonneland et al., “Alcohol Intake, Drinking Patterns and Risk of Postmenopausal Breast Cancer in Denmark: A Prospective Cohort Study,” Cancer Causes Control 14: 277-284 (2003)). Therefore, limiting exposure to estrogens and reducing the overall number of menstrual cycles in one's lifetime through dietary and lifestyle changes can be the simplest means to reduce disease risk. In particular, the identification of dietary compounds that have estrogen-reducing effects holds great promise in developing chemopreventive strategies to abrogate risk of these diseases, especially endometriosis and conditions directly related to endometriosis. This provides a new route to the treatment of endometriosis. Such new treatment methods can employ previously known compounds, but does so for uses not previously envisioned.

Recent studies have demonstrated that the eutopic (normal endometrium within the uterine cavity) and ectopic endometrium (endometrium growing outside of the uterine cavity) of women with endometriosis expresses the enzyme aromatase whilst aromatase expression is not detected in the endometrium of women without disease. Moreover, the ectopic endometrium has decreased expression of 17β-hydroxysteroid dehydrogenase type II (17β-HSD-II), the enzyme responsible for the metabolism of estradiol to estrone, compared to eutopic endometrium. Taken together these findings indicate that ectopic endometrium can produce estrogen from androgens and has an impaired capacity to metabolize estradiol compared to eutopic endometrium thus resulting in increased local estrogen concentrations. Therefore, changes in aromatase regulation and estrogen production in endometrial cells are a potentially useful diagnostic marker and a therapeutic target.

Molecular mechanisms regulating the expression and activity of aromatase in human endometrium has been shown to involve the interplay of two transcription factors, chicken ovalbumin upstream promoter-transcription factor (COUP-TF) and steroidogenic factor-I (SF-I). COUP-TF inhibits aromatase expression in the eutopic endometrium whereas SF-I stimulates aromatase mRNA and protein expression and is present in the ectopic endometrium of women with endometriosis. Both COUP-TF and SF-I compete for the nuclear receptor half site in the aromatase gene promoter. It is thought that in women with endometriosis, increased levels of SF-I displace COUP-TF from the P450_{AROM} promoter thus removing the “brake” on aromatase expression. The result is increased transcription of aromatase that ultimately leads to increased estrogen production in the endometrium. Currently there is little information available regarding the regulation of aromatase and SF-I in endometriosis, however, it has been reported that SF-I and P450_{AROM} mRNA and protein expression in the ectopic endometrium can be induced by prostaglandin E₂ (PGE₂) but not interleukins or other prostanoids. Therefore increased prostaglandin bioavailability may play a role in the inappropriate estrogen production by ectopic endometrium. Bioavailability of primary prostaglandins depends on the balance between prostaglandin synthesis and metabolism. The primary prostaglandins, PGE₂ and PGF_{2α} are formed from arachidonic acid by prostaglandin H synthase (PGHS) also known as cyclooxygenase (COX). Two isoforms of COX have been identified in human endometrium, the constitutively expressed form, COX-I and the inducible form, COX-II. Prostaglandins are metabolized by a NAD⁺-dependent 15-hydroxyprostaglandin dehydrogenase (PGDH), which catalyzes the conversion of PGE₂ and PGF_{2□} to their biologically inactive 15-keto derivatives. Estrogens have been demonstrated to enhance the expression and activity of PGHS-II resulting in increased PGE₂ production. Therefore, although Applicant does not intend to be bound by this theory, it is proposed that a feed forward mechanism of inappropriate estrogen production in endometriosis is driven by dysfunction of the mechanisms regulating PGE₂ bioavailability.

The first line management of endometriosis related pelvic pain commonly involves the use of oral contraceptives. Many women continue to experience pelvic pain on this therapy and are therefore tried on other medications including progestins, danazol and GnRH agonists. Each of these agents suppresses endometrial proliferation and reduces pelvic pain in a majority of women with endometriosis. However, their use is restricted by their side effects. The long-term use of progestins is limited by concerns related to adverse lipid changes, depression, fluid retention, and breast tendemess. Danazol has significant androgenic actions including acne, hirsutism, male pattern hair loss and changes to voice and body habitus. Some of these masculinizing side effects are irreversible and thus severely limit its use in women. Because of concerns regarding hypoestrogenemia induced bone loss, the FDA has limited approval of GnRH agonist use to a single six-month course. Other highly significant problems with these medications that limit their acceptability to patients include hot flashes, depression and vaginal dryness. A further problem with all of these medical treatments of endometriosis is that they either significantly inhibit or contraindicate pregnancy. Thus women with endometriosis suffering from both infertility and chronic pelvic pain have to choose between fertility therapies and management of their pelvic pain. Despite the initial success seen with an aromatase inhibitor in the treatment of endometriosis it is expected that these agents will induce hypoestrogenemic states similar to existing therapeutic agents that will limit the potential application of these drugs to post-menopausal women with recalcitrant endometriomas. Therefore, Applicant has identified novel therapeutic agents that will effectively inhibit aromatase activity in ectopic endometrium while preserving fertility in women of reproductive age. These therapeutic agents are described in further detail below.

According to this hypothesis, treatment with these therapeutic agents attenuate COX-II and aromatase expression (mRNA and protein) and activity in endometrial cells from women with endometriosis.

Additionally, the use of compounds and compositions that exert an anti-inflammatory effect and an effect that inhibits the activity of cellular adhesion molecules also provides a new route to the treatment of endometriosis. In particular, inhibition of adherence to new surfaces by cells migrating out of the uterus would exert an effect that would inhibit the progression of the disease process of endometriosis and would diminish the symptoms of the disease.

Studies show that Japanese women have longer menstrual cycle lengths (greater than the 28 day average) and lower circulating estrogen levels compared to Western populations (H. Shimizu et al., “Serum Oestrogen Levels in Postmenopausal Women: Comparison of American Whites and Japanese in Japan,” Br. J. Cancer 62: 451-453 (1990); H. Olsson et al., “Retrospective Assessment of Menstrual Cycle Length in Patients with Breast Cancer, in Patients with Benign Breast Disease, and in Women Without Breast Disease,” J. Natl. Cancer Inst. 70: 17-20 (1983); T. J. Key et al., “Sex Hormones in Women in Rural China and in Britain,” Br. J. Cancer 62: 631-636 (1990), which until now has been at least partly attributed to the increased intake of soy protein among Asian populations (L. J. Lu et al., “Decreased Ovarian Hormones During a Soya Diet: Implications for Breast Cancer Protection,” Cancer Res. 60: 4112-4121 (2000); K. D. Setchell et al., “Nonsteroidal Estrogens of Dietary Origin: Possible Roles in Hormone-Dependent Disease,” Am. J. Clin. Nutr. 40: 569-578 (1984); A. Cassidy et al., “Biological Effects of a Diet of Soy Protein Rich in Isoflavones on the Menstrual Cycle of Premenopausal Women,” Am. J. Clin. Nutr. 60: 333-340 (1994)). Another less explored component but main staple of the Japanese diet is seaweed, which accounts for approximately 10-25% of their food intake (Y. Kagawa, “Impact of Westernization on the Nutrition of Japanese: Changes in Physique, Cancer, Longevity and Centinarians,” Prev. Med. 7: 205-217 (1978); C. G. Wood, “Seaweed Extracts: A Unique Ocean Resource,” J. Chem. Educ. 51: 449-452 (1974)). Other reported estimated daily intakes are as high as 3-13 g/day (J. Teas et al., “Algae—A Poor Man's HAART,” Med. Hypotheses 62: 507-510 (2004)) A major source of dietary seaweed among Japanese populations is the edible brown kelp, wakame (Undaria pinnatifida) and kombu (Laminaria japonica). These species and the Atlantic brown kelp, bladderwrack (Fucus vesiculosus), have been shown to exert powerful anti-hypertensive activity related to angiotensin-I-converting enzyme inhibition (M. Sato et al., “Antihypertensive Effects of Hydrolysates of Wakame (Undaria pinnatifida) and Their Angiotensin-I-Converting Enzyme Inhibitory Activity,” Ann. Nutr. Metab. 46: 259-267 (2002)), to possess antibacterial and antioxidant properties related to their high polyphenolic content (S. Abdussalam, “Drugs from Seaweeds,” Med. Hypotheses 32: 33-35 (1990)), and to prevent dioxin absorption and accelerate dioxin excretion in rats (K. Morita & T. Nakano, “Seaweed Accelerates the Excretion of Dioxin Stored in Rats,” J. Agric. Food Chem. 50: 910-917 (2002)). Other chemopreventive properties such as antiviral activity (M. Luscher-Mattli, “Polyanions—A Lost Chance in the Fight Against HIV and Other Virus Diseases,” Antivir. Chem. Chemother. 11: 249-259 (2000); D. J. Schaeffer & V. S. Krylov, “Anti-HIV Activity of Extracts and Compounds from Algae and Cyanobacteria,” Ecotoxicol. Environ. Saf. 45: 208-227 (2000)), immunostimulatory effects (R. Cooper et al., “GFR, a Preparation of Tasmanian Undaria pinnatifida Is Associated with Healing and Inhibition of Reactivation of Herpes,” BMC Complement Altern. Med. 2: 11 (2002)), anti-proliferative effects on 7,12-dimethylbenz(a)-anthracene-induced rat mammary tumors (H. Funahashi et al., “Wakame Seaweed Suppresses the Proliferation of 7,12-Dimethylbenz(a)-anthracene-lnduced Mammary Tumors in Rats,” Jpn. J. Cancer Res. 90: 922-927 (1999); J. Teas et al., “Dietary Seaweed (Laminaria) and Mammary Carcinogenesis in Rats,” Cancer Res. 44: 2758-2761 (1984)), and anti-tumor and anti-metastatic activities in xenograft mouse models (S. Koyanagi et al., “Oversulfation of Fucoidan Enhances Its Anti-Angiogenic and Antitumor Activities,” Biochem. Pharmacol. 65: 173-179 (2003)), have been associated with the high level of sulfated polysaccharides, also known as fucoidans, found in brown seaweed. This suggests a new way of treating endometriosis that is safe and is effective. This new way of treating endometriosis directly treats the hormonal component of the disease.

Intake of bladderwrack, as well as other brown kelp species, also has been shown to alter cholesterol metabolism and to significantly lower plasma cholesterol levels (J. Ara et al., “Hypolipidaemic Activity of Seaweed from Karachi Coast,” Phytother. Res. 16: 479-483 (2002); T. Kaneda et al., “Studies on the Effects of Marine Products on Cholesterol Metabolism, 1. The Effects of Edible Seaweed (Bull. Jap. Soc. Sci. Fish. 29: 1020-1025 (1963)). A possible mechanism of action involves competitive inhibition by fucosterols found in kelp. Since cholesterol is the precursor involved in steroid hormone biosynthesis, a reduction in cholesterol bioavailability could lower circulating plasma 17β-estradiol levels that can lead to alterations in menstrual cycling patterns in pre-menopausal women. Until now, no studies have been performed in humans to determine the effects of brown kelp on menstrual cycling patterns and sex hormone status in pre-menopausal women, particularly in women with or at risk for estrogen-dependent diseases.” (C. F. Skibola, “The Effect of Fucus vesiculosus, an Edible Brown Seaweed, on the Menstrual Cycle Length and Hormonal Status in Three Pre-Menopausal Women: A Case Report” BMC Complement. Altern. Med. 4: 10 (2005)) (“C. F. Skibola (2005)”).

Rates of estrogen-dependent cancers are among the highest in Western countries and lower in the East. These variations can be attributable to differences in dietary exposures such as higher seaweed consumption among Asian populations. The edible brown kelp, Fucus vesiculosus (bladderwrack), as well as other brown kelp species, has been found to lower plasma cholesterol levels. Since cholesterol is a precursor to sex hormone biosynthesis, kelp consumption can alter circulating sex hormone levels and menstrual cycling patterns. In particular, dietary kelp can be beneficial to women with or at high risk for estrogen-dependent diseases. To test this, bladderwrack was administered to three pre-menopausal women with abnormal menstrual cycling patterns and/or menstrual-related disease histories. (C. F. Skibola (2005), supra).

Intake of bladderwrack was associated with significant increases in menstrual cycle lengths, ranging from an increase of 5.5 to 14 days. In addition, hormone measurements ascertained for one woman revealed significant anti-estrogenic and progestagenic effects following kelp administration. These pilot data suggest that dietary bladderwrack can prolong the length of the menstrual cycle and exert anti-estrogenic effects in pre-menopausal women. Further, these studies also suggest that seaweed can be another important dietary component apart from soy that is responsible for the reduced risk of estrogen-related cancers observed in Japanese populations. (C. F. Skibola (2005), supra).

However, there has been no identification of the compound or compounds present in bladderwrack that are responsible for the anti-estrogenic activity or for the alleviation of the symptoms of estrogen-dependent diseases and conditions, especially endometriosis. In particular, there exists no formulation that is specifically formulated for the treatment of estrogen-dependent diseases and conditions, especially endometriosis, and that provides convenient administration of the compound or compounds in a substantially purified form.

Accordingly, one aspect of the invention comprises a pharmaceutical composition for the treatment of an estrogen-dependent disease or condition, the composition comprising:

(1) at least one polysaccharide selected from the group consisting of an alginate and a fucoidan in a quantity effective to treat an estrogen-dependent disease or condition; and

(2) a pharmaceutically acceptable carrier.

When the polysaccharide is an alginate, it typically comprises a polymer of guluronic acid and mannuronic acid. Alginates are linear unbranched polymers containing β-(1→4)-linked D-mannuronic acid (M) and α-(1→4)-linked L-guluronic acid (G) residues. Although these residues are epimers (D-mannuronic acid residues being enzymatically converted to L-guluronic after polymerization) and only differ at C5, they possess very different conformations; D-mannuronic acid being ⁴C₁ with diequatorial links between them and L-guluronic acid being ¹C₄ with diaxial links between them. Bacterial alginates are additionally O-acetylated on the 2 and/or 3 positions of the D-mannuronic acid residues. The bacterial O-acetylase may be used to O-acetylate the algal alginates, so increasing their water binding.

Alginates are not random copolymers, but, according to the source of the alginate, consist of blocks of similar or strictly alternating residues, such as MMMMMM, GGGGGG, or GMGMGM, each of which has different conformations and reactivity. For example, the M/G ratio of alginate from Macrocystis pyrifera is about 1.6 whereas that from Laminaria hyperborea is about 0.45 Alginates can be prepared with a wide range of molecular weights from about 50 to about 1×10⁵ Daltons; for compositions according to the present invention, the molecular weights typically range from about 5000 Daltons to about 50,000 Daltons.

Poly β-(1→4)-linked D-mannuronate prefers forming a 3-fold left-handed helix with (weak) intra-molecular hydrogen bonding between the hydroxyl group in the 3 position and the subsequent ring oxygen (i.e. O3-H→O′). Poly α-(1→4)-linked L-guluronate forms stiffer (and more acid-stable) 2-fold screw helical chains, preferring intra-molecular hydrogen bonding between the carboxyl group and the 2-OH group of the prior residues and (weaker) the 3-OH group of the subsequent residues. The diaxial links also inherently allow less flexibility. Alternating poly α-(1→4)-linked L-guluronate-β-(1→4)-linked D-mannuronate contains both equatorial-axial and axial-equatorial links and take up dissimilar rather disorderly conformations. They have hydrogen bonds between the carboxyl group on the mannuronate and the 2-OH and 3-OH groups of the subsequent guluronate but the differing degrees of freedom of the two residues gives greater overall flexibility than the poly β-(1→4)-linked D-mannuronate chains. The free carboxylic acids (without counterion) have a water molecule H₃O⁺ firmly hydrogen bound to carboxylate (pKₐ M 3.38, pKₐ G 3.65). Ca²⁺ ions can replace this hydrogen bonding, zipping guluronate, but not mannuronate, chains together stoichiometrically in a supposedly egg-box like conformation (the ions being the eggs in the puckered box formed by sequential saccharides; the box possibly consists of six oxygen ligands from the 2-OH and 3-OH plus a carboxylate oxygen of the subsequent residue, supplied by each poly-guluronate chain) with 7^{th} and 8^{th} ligands from the (1→4)—O-linkages slightly further away. The chains are stabilized by hydrogen bonding between the other carboxylate oxygen and 2-OH groups on the subsequent residues. Poly-guluronate has specific binding sites for calcium consisting of five oxygen ligands from the 2-OH and 3-OH, (1→4)-O-linkage and carboxylate and ring oxygen of the subsequent residue, so holding the calcium ready for this junction zone formation. This junction zone optimally requires 10-12 residues (depending on parameterization) to form half a complete revolution (as optimized using the AMBER-96 force field) of the parallel left-handed double helix (see below) and consequent permanent junction zone formation. Interactions with further poly-guluronate segments favor an unwound sheet-like structure; the winding-unwinding only requiring changes in the anomeric linkage angles (φ and Ψ) of about 10° while retaining the hydrogen bonding and ionic linkages. A possibly-related two-stage junction zone formation has been recently proposed to occur in alginic acid gels, based on X-ray scattering and rheological characterization. Curiously, calcium poly-guluronate also forms a (only slightly less) stable parallel right-handed helix (φ and Ψ) further changing by about 10°) of about the same number of residues per helix where the calcium ions sit in a pocket approximately equispaced from 10 oxygen ligands (from the 2-OH and 3-OH, (1→4)-O-linkage and a carboxylate and ring oxygen of the subsequent residue from both chains) and where hydrogen bonds are found from alternative carboxyl groups and both the prior 2-OH group and the 3-OH group of the prior residues on the parallel strand. Under similar conditions, poly-mannuronic acid blocks take up a less-gelling ribbon conformation, where carboxylate groups on sequential residues may bind calcium intra- or inter-molecularly.

Alginic acid in brown seaweeds is mainly present as calcium, magnesium, or sodium salts. The first step in the manufacture of alginate is to convert the relatively insoluble calcium and magnesium alginate by ion exchange under alkaline conditions. Typically, the alginophyte is first treated with dilute mineral acid before alkali extraction to facilitate the ion exchange process. The crude sodium alginate solution extracted is then filtered and precipitated with divalent calcium ions to form the insoluble calcium salt. The latter, on separation, is converted to insoluble alginic acid by acidification to accomplish the removal of calcium ions. Then the alginic acid gels, after dehydration, are mixed with Na₂CO₃ in powder form to convert the alginic acid to soluble sodium salt again. Finally, the sodium alginate pastes formed are dried and milled into sodium alginate powder.

The manufacture of sodium alginate by the calcification process involves pretreatment, hot extraction, dilution, crude filtration, flotation, fine filtration, calcification, bleaching, acidification, dehydration, incorporation, drying, and milling. For pretreatment, the alginophytes, such as Laminaria sp., are treated first with 0.1-0.4% commercial formalin solution at room temperature for several hours to fix the pigments together with the phenolic substances present in the thalli to diminish the coloration of the extracted liquor. Then, the thalli are soaked with dilute acid such as 0.1 N sulfuric acid or hydrochloric acid for 30 minutes at room temperature to convert salts of alginate into alginic acid. For hot extraction, the treated wet thalli are extracted with 1% sodium carbonate solution at about 50-60° C. for Laminaria and about 75° C. for Sargassum for 1-2 hours in a steam-jacketed cooker equipped with a stirrer. The concentration of alginate in extracted liquor is about 1%. This extracted liquor is too viscous to filter because of the high viscosity of the alginates; typically, the extracted liquor is diluted with 4-6 volumes of water to about 0.2-0.3% concentration (with a viscosity of about 20-100 Cp). The crude filtration step is done with a rotary filter fixed with a 30-40 mesh nylon screen. In flotation, air is forced into the crude filtrate in tanks, and the bubbles generated adhere to the fine particles of insoluble residue to form flocs, floating on the surface with the bubbles. After the flotated mixture is allowed to stand for several hours, the clarified liquor beneath the surface is drawn off at the bottom of the tanks. Fine filtration is then performed, particularly if food grade alginate is desired. For fine filtration, the clarified liquor is filtered with a rotary nylon screen (100-120 mesh) filter or with the Dorr-Oliver rotary filter, coated with filter aid to remove the dispersed small particles. For calcification, the filtrate is calcified with calcium chloride solution to precipitate the calcium alginate. For bleaching, the calcium alginate gels formed are bleached with sodium hypochlorite (effective chlorine 0.05-0.10%). Calcium alginate is more resistant to degradation than alginic acid. For acidification, the bleached calcium alginate gels are treated with dilute sulfuric acid or hydrochloric acid solution (0.5 N) to convert calcium alginate into alginic acid, typically by a three-step counter-current conversion. For dehydration, after washing with water, the gels are sent to a hydraulic press or screw press to dewater the gels with the solids content reaching at least 25%. For incorporation, the alginic acid gels are incorporated with sodium carbonate powder in a mixer. The pastes formed are squeezed through a porous plate, and the extrusions are chopped into pellets. For the final drying and milling step, the pellets are conveyed into a drying chamber or a fluid-bed dryer with a vibrator to dry at 80° C., and then milled to sodium alginate powder (60 mesh).

In an alternative process for preparation of sodium alginate, calcification is not performed. In this alternative, after fine filtration the filtrate is acidified with dilute sulfuric acid or hydrochloric acid in a pipeline controlled with a pH meter so that the pH is about 1.5-2.0, and alginic acid gels are precipitated and float to the liquid surface by CO₂ bubbles formed by neutralization of acid and the excess of alkaline extractant. The mixture is allowed to stand for 1 hour, allowing completion of the reaction and flotation of alginic acid. The alginic acid gels are then filtered with nylon bags and dewatered by hydraulic press, basket centrifuge, or screw press. The alginic acid gels typically contain about 20-25% solids. The gels are conveyed to a conversion tank in which ethyl alcohol, sodium hydroxide (40%) and bleaching solution (sodium hypochlorite) are added. The alginic acid is converted to the sodium salt in ethyl alcohol, and at the same time is bleached. The used ethyl alcohol is removed by using a basket-type centrifuge and pumped to a recovery facility. The fibrous sodium alginate formed is then sent to a drying chamber which is equipped for the recovery of alcohol vapor. The dried product is then ground to sodium alginate powder.

Fucoidan is a generic term for sulfated polysaccharides found in the cell-wall matrix of brown algae. A particular useful source of fucoidan is Fucus vesiculosus, but other brown algae are also sources of fucoidans. Fucoidans useful in methods and compositions according to the present invention are not limited to those isolated, extracted, or partially purified from Fucus vesiculosus, but include fucoidans isolated, extracted, or partially purified from other algae or chemically synthesized. Brown algae (seaweed) contains about 4% of fucoidans. The fucoidans are present as F-fucoidan, which is a polymer that contains primarily sulfated fucose, and U-fucoidan, which contains about 20% of glucuronic acid. Fucoidans are further described in U.S. Pat. No. 5,948,405 to Cedro et al., and in U.S. Pat. No. 6,028,191 to Nardella et al., both incorporated herein by this reference. The use of extracts of Fucus vesiculosus is described in U.S. Pat. No. 5,508,033 to Briand, incorporated herein by this reference. Alternatively, compositions according to the present invention can further include fucose and/or derivatives of fucose.

As used herein, recitation of components that can be purified from algae or other plants, such as, but not limited to, alginates and fucoidans, is intended to refer to ingredients that are partially or completely purified from the algae or other plants. Alternatively these components can be administered in the form of extracts, powders, or plant parts as long as the components are administered in a form in which the activity of the alginates, fucoidans, and other active ingredients is standardized. The term “partially or completely purified,” as used herein, means that the component exists in a state in which other molecules that normally occur together with it in the plant from which the component is purified have been partially or completely removed.

Typically, the composition comprises a quantity of polysaccharide such that the daily dose of the polysaccharide that is administered is from about 10 mg to about 3000 mg. Preferably, the composition comprises a quantity of polysaccharide such that the daily dose of the polysaccharide that is administered is from about 100 mg to about 1500 mg. This daily dose could be administered in one or more dosage units. The term “dosage unit” as used herein, refers to a physical dosage form, such as, but not limited to, a pill, tablet, or capsule. Other physical dosage forms can be used and are described below.

The composition typically includes both an alginate and a fucoidan. However, in alternatives, the composition can include only an alginate or only a fucoidan. It is generally preferred, though, to include both an alginate and a fucoidan. Although Applicant does not intend to be bound by this theory, it is believed that the fucoidans are active on their own, and the alginates are synergistic with the fucoidans. However, the alginates can also have activity independently.

Optionally, the fucoidans and alginates can be modified by additional sulfation or methylation. Methods for modification of fucoidans or alginates by sulfation or methylation are known in the art, and are described, for example, in S. Koyanagi et al., “Oversulfation of Fucoidan Enhances Its Anti-Angiogenic and Antitumor Activities,” Biochem. Pharmacol. 65: 173-179 (2003) for sulfation and in R. T. Morrison & R. N. Boyd, Organic Chemistry (5^{th} ed., Allyn & Bacon, Boston, 1987), pp. 1310 for methylation. Methylation of the hydroxyl groups of a polysaccharide can be accomplished, for example, by reaction with methyl sulfate in a basic environment (a modification of the Williamson ether synthesis).

Other algae could be used to provide fucoidans or alginates. For example, they can be obtained from Cladosiphon okamuranus, grown in Japan, or from Ascophyllum nodosum, grown in Norway. Fucoidan from Cladosiphon okamuranus has a typical structure based on a backbone of L-fucose; fucoidan from Ascophyllum nodosum has a different structure. Alternatively, as indicated above, they can be obtained by chemical synthetic methods known in the art for synthesis of polysaccharides and substituted polysaccharides.

In another alternative, the fucoidans or alginates could be obtained by bacterial fermentation.

In still another alternative, the fucoidans or alginates could be obtained from animal sources, such as the jelly coat of sea urchin eggs or sea cucumber.

Compositions according to the present invention can include other ingredients. For example, polymers of glucaric acid, such as calcium-D-glucarate, can be included in a quantity sufficient to inhibit the enzyme β-glucuronidase. The enzyme β-glucuronidase interferes with the estrogen-binding activity of glucuronic acid. However, forms of glucaric acid, including calcium D-glucarate, inhibit this enzyme, thus allowing the guluronic acid to continue to bind estrogen without interference from the enzyme β-glucuronidase. Typically, if calcium D-glucarate is included in the composition, the quantity of calcium D-glucarate is such that the daily dose of the calcium D-glucarate is from about 50 mg to about 5 g; preferably, the quantity of calcium D-glucarate is such that the daily dose of the calcium D-glucarate is from about 100 mg to about 3000 mg. As indicated above, this can be included in one or more dosage units.

Alternatively, other glucaric acid derivatives, such as D-glucaro-1,4-lactone, can be used.

Compositions according to the present invention can also include diindolylmethane (DIM) and/or indole-3-carbinol. These compounds act as estrogen inhibitors. Typically, if diindolylmethane or indole-3-carbinol are included in compositions according to the present invention, the quantity of diindolylmethane in the composition is such that the daily dose of the diindolylmethane is from about 25 mg to about 2 g, and the quantity of indole-3-carbinol in the composition is such that the daily dose of the indole-3-carbinol is from about 50 mg to about 2 g. Preferably, the quantity of diindolylmethane in the composition is such that the daily dose of the diindolylmethane is from about 25 mg to about 1000 mg, and the quantity of indole-3-carbinol in the composition is such that the daily dose of the indole-3-carbinol is from about 100 mg to about 1000 mg. As indicated above, these dosages can be included in one or more dosage units.

Other ingredients can also be used. For example, the composition can include anastrozole in a quantity sufficient to inhibit aromatase. Other aromatase inhibitors can alternatively be used. Additional aromatase inhibitors include, but are not limited to, formestane, exemestane, vorozole, and letrozole.

The composition can also include a progestin or progesterone in a quantity sufficient to inhibit the effects of estrogen. The progestin or progesterone can be selected from the group consisting of megestrol acetate, medroxyprogesterone acetate, 19-nortestosterone, norethindrone, ethynodiol diacetate, norgestrel, desogestrel, norgestimate, and their derivatives.

The composition can also include lignans or sterols from algal sources. These compounds can act as antagonists for the binding of estrogen to estrogen receptors and thus block the activity of naturally-occurring estrogens. If lignans or sterols from algal sources are included, the composition contains a quantity sufficient to act as an antagonist for the binding of estrogen to estrogen receptors.

Another aspect of the invention is a composition comprising:

(1) calcium D-glucarate in a quantity sufficient to treat an estrogen-dependent condition;

(2) diindolylmethane in a quantity sufficient to treat an estrogen-dependent condition; and

(3) a pharmaceutically acceptable carrier.

In this alternative, without polysaccharides, typically the composition comprises a quantity of calcium D-glucarate such that the daily dose of calcium D-glucarate is from about 100 mg to about 3000 mg, and the composition comprises a quantity of diindolylmethane such that the daily dose of diindolylmethane is from about 100 mg to about 3000 mg. As indicates above, these dosages can be included in one or more dosage units.

This alternative composition, without polysaccharides, can also include other components as described above, including, but not limited to, an aromatase inhibitor such as anastrozole or another aromatase inhibitor, progesterone or a progestin, a lignan from an algal source, or a steroid from an algal source.

Typically, compositions according to the present invention possess the activity of inhibiting the expression of at least one protein selected from the group consisting of aromatase, SF-I, COX-I, COX-I, and 15-hydroxyprostaglandin dehydrogenase at the level of transcription.

Accordingly, another aspect of the present invention is a method of treating an estrogen-dependent disease or condition comprising administering an effective quantity of a composition according to the present invention, as described above, to an individual suffering from such a disease or condition, as described above. The estrogen-dependent disease or condition can be endometriosis.

Methods according to the present invention can be used to treat both humans and non-human mammals that have an estrogen-dependent disease or condition. Non-human mammals suitable for treatment include socially or economically important animals selected from the group consisting of dogs, cats, horses, sheep, pigs, cows, and goats. Methods according to the present invention are not limited to the treatment of humans.

Pharmaceutically acceptable carriers suitable for use in compositions according to the present invention can include, but are not necessarily limited to, calcium carbonate, calcium phosphate, various sugars or types of starch, cellulose derivatives, gelatin, vegetable oils, polyethylene glycols and physiologically compatible solvents. Additional pharmaceutically acceptable carriers include, but are not limited to, any and/or all of solvents, including aqueous and non-aqueous solvents, dispersion media, coatings, antibacterial and/or antifungal agents, isotonic and/or absorption delaying agent, and/or the like. The use of such media and/or agents for pharmaceutically active substances is well known in the art. Except insofar as any conventional medium, carrier, or agent is incompatible with the active ingredient or ingredients, its use in a composition according to the present invention is contemplated. Supplementary active ingredients can also be incorporated into the compositions, especially as described below under combination therapy. For administration of any of the compounds used in the present invention, preparations should meet sterility, pyrogenicity, general safety, and purity standards as required by the FDA Office of Biologics Standards or by other regulatory organizations regulating drugs.

Thus, compositions according to the invention can be formulated for oral, sustained-release oral, buccal, sublingual, inhalation, insufflation, or parenteral administration. However, formulation for oral administration is typically preferred.

If a composition according to the present invention is formulated for oral administration, either in a conventional or a sustained-release preparation, it is typically administered in a conventional unit dosage form such as a tablet, a capsule, a pill, a troche, a wafer, a powder, or a liquid such as a solution, a suspension, a tincture, or a syrup. Oral formulations typically include such normally employed excipients as, for example, pharmaceutical grades of mannitol, lactose, starch, magnesium stearate, sodium saccharin, cellulose, magnesium carbonate, and other conventional pharmaceutical excipients. In certain defined embodiments, oral pharmaceutical compositions will comprise an inert diluent and/or assimilable edible carrier, and/or they may be enclosed in hard or soft shell gelatin capsules. Alternatively, they may be compressed into tablets. As another alternative, particularly for veterinary practice, they can be incorporated directly into food. For oral therapeutic administration, they can be incorporated with excipients or used in the form of ingestible tablets, buccal tablets, dragees, pills, troches, capsules, wafers, or other conventional dosage forms.

The tablets, pills, troches, capsules, wafers, or other conventional dosage forms can also contain the following: a binder, such as gum tragacanth, acacia, cornstarch, sorbitol, mucilage of starch, polyvinylpyrrolidone, or gelatin; excipients or fillers such as dicalcium phosphate, lactose, microcrystalline cellulose, or sugar; a disintegrating agent such as potato starch, croscarmellose sodium, or sodium starch glycolate, or alginic acid; a lubricant such as magnesium stearate, stearic acid, talc, polyethylene glycol, or silica; a sweetening agent, such as sucrose, lactose, or saccharin; a wetting agent such as sodium lauryl sulfate; or a flavoring agent, such as peppermint, oil of wintergreen, orange flavoring, or cherry flavoring. When the dosage unit form is a capsule, it can contain, in addition to materials of the above types, a liquid carrier. Various other materials can be present as coatings or to otherwise modify the physical form and properties of the dosage unit. For instance, tablets, pills, or capsules can be coated with shellac, sugar, or both. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

In one alternative, a sustained-release formulation is used. Sustained-release formulations are well-known in the art. For example, they can include the use of polysaccharides such as xanthan gum and locust bean gum in conjunction with carriers such as dimethylsiloxane, silica acid, a mixture of mannans and galactans, xanthans, and micronized seaweed, as recited in U.S. Pat. No. 6,039,980 to Baichwal, incorporated herein by this reference. Other sustained-release formulations incorporate a biodegradable polymer, such as the lactic acid-glycolic acid polymer recited in U.S. Pat. No. 6,740,634 to Saikawa et al., incorporated herein by this reference. Still other sustained-release formulations incorporate an expandable lattice that includes a polymer based on polyvinyl alcohol and polyethylene glycol, as recited in U.S. Pat. No. 4,428,926 to Keith, incorporated herein by this reference. Still other sustained-release formulations are based on the Eudragit™ polymers of Rohm & Haas, that include copolymers of acrylate and methacrylates with quaternary ammonium groups as functional groups as well as ethylacrylate methylmethacrylate copolymers with a neutral ester group. Other extended release formulations are known in the art.

Oral liquid preparations can be in the form of, for example, aqueous or oily suspensions, solutions, emulsions, syrups, tinctures, or elixirs, or can be presented as a dry product for reconstitution with water or other suitable vehicles before use. Such liquid preparations can contain conventional additives such as suspending agents, for example, sorbitol syrup, methylcellulose, glucose/sugar syrup, gelatin, hydroxymethylcellulose, carboxymethylcellulose, aluminum stearate gel, or hydrogenated edible fats; emulsifying agents, such as lecithin, sorbitan monooleate, or acacia; non-aqueous vehicles (which may include edible oils), for example, almond oil, fractionated coconut oil, oily esters, propylene glycol, or ethyl alcohol; or preservatives, for example, methylparaben, propylparaben, or sorbic acid. The preparations can also contain buffer salts, flavoring, coloring, or sweetening agents (e.g., mannitol) as appropriate.

One skilled in the art recognizes that the route of administration is an important determinant of the rate of efficiency of absorption. For example, the alimentary route, e.g., oral, rectal, sublingual, or buccal, is generally considered the safest route of administration. The delivery of the drugs into the circulation is slow, thus eliminating rapid high blood levels of the drugs that could potentially have adverse acute effects. Although this is considered the safest route of administration, there are several disadvantages. One important disadvantage is that the rate of absorption varies, which is a significant problem if a small range in blood levels separates a drug's desired therapeutic effect from its toxic effect, i.e., if the drug has a relatively low therapeutic index. Also, patient compliance is not always ensured, especially if the rectal route of administration is chosen or if oral administration is perceived by the patient as unpleasant. Furthermore, with oral administration, extensive hepatic metabolism can occur before the drug reaches its target site. Another route of administration is parenteral, which bypasses the alimentary tract. One important advantage of parenteral administration is that the time for the drug to reach its target site is decreased, resulting in a rapid response, which is essential in an emergency. Furthermore, parenteral administration allows for delivery of a more accurate dose. Parenteral administration also allows for more rapid absorption of the drug, which can result in increased adverse effects. Unlike alimentary administration, parenteral administration requires a sterile formulation of the drug and aseptic techniques are essential. The most significant disadvantage to parenteral administration is that it is not suitable for insoluble substances. In addition to alimentary and parenteral administration routes, topical and inhalation administrations can be useful. Topical administration of a drug is useful for treatment of local conditions; however, there is usually little systemic absorption. Inhalation of a drug provides rapid access to the circulation and is the common route of administration for gaseous and volatile drugs, or drugs that can be vaporized or nebulized. It is also a desired route of administration when the targets for the drug are present in the pulmonary system.

When compounds are formulated for parenteral administration, e.g., formulated for injection via the intravenous, intramuscular, subcutaneous, intralesional, or intraperitoneal routes, many options are possible. The preparation of an aqueous composition that contains an effective amount of the β-adrenergic inverse agonist as an active ingredient will be known to those of skill in the art. Typically, such compositions can be prepared as injectables, either as liquid solutions and/or suspensions. Solid forms suitable for use to prepare solutions and/or suspensions upon the addition of a liquid prior to injection can also be prepared. The preparations can also be emulsified.

The pharmaceutical forms suitable for injectable use include sterile aqueous solutions and/or dispersions; formulations including sesame oil, peanut oil, synthetic fatty acid esters such as ethyl oleate, triglycerides, and/or aqueous propylene glycol; and/or sterile powders for the extemporaneous preparation of sterile injectable solutions and/or dispersions. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. In all cases the form must be sterile and/or must be fluid to the extent that the solution will pass readily through a syringe and needle of suitable diameter for administration. It must be stable under the conditions of manufacture and storage and must be preserved against the contaminating action of microorganisms, such as bacteria or fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and/or mixtures thereof and/or in oils. Under ordinary conditions of storage and use, these preparations contain a preservative to prevent the growth of microorganisms. Suitable non-sensitizing and non-allergenic preservatives are well known in the art.

The carrier can also be a solvent and/or dispersion medium containing, for example, water, ethanol, a polyol (for example, glycerol, propylene glycol, and/or liquid polyethylene glycol, and/or the like), suitable mixtures thereof, and/or vegetable oils. The proper fluidity can be maintained for example, by the use of a coating, such as lecithin, by the maintenance of a suitable particle size in the case of a dispersion, and/or by the use of surfactants. The prevention of the action of microorganisms can be brought about by the inclusion of various antibacterial and/or antifungal agents, for example, parabens, chlorobutanol, phenol, sorbic acid, or thimerosal. In many cases it will be preferable to include isotonic agents, for example, sugars or sodium chloride. In many cases, it is preferable to prepare the solution in physiologically compatible buffers such as Hanks's solution, Ringer's solution, or physiological saline buffer. Prolonged absorption of the injectable compositions can be brought about by the use in the compositions of agents delaying absorption, for example, aluminum monostearate and/or gelatin.

Sterile injectable solutions are prepared by incorporating the active compounds in the required amount in the appropriate solvent with various of the other ingredients enumerated above, as required, followed by sterilization. Sterilization is typically performed by filtration. Generally, dispersions are prepared by incorporating the various sterilized active ingredients into a sterile vehicle which contains the basic dispersion medium and/or the other required ingredients. In the case of sterile powders for the preparation of sterile injectable solutions, the preferred methods of preparation are vacuum-drying and/or freeze-drying techniques that yield a powder of the active ingredients plus any additional desires ingredients from a previously sterile-filtered solution thereof. The preparation of more-concentrated or highly-concentration solutions for direct injection is also contemplated, where the use of dimethyl sulfoxide (DMSO) as solvent is envisioned to result in extremely rapid penetration, delivering high concentrations of the active agents to a small area if desired.

For parenteral administration in an aqueous solution, for example, the solution should be suitably buffered if necessary and/or the liquid diluent first rendered isotonic with sufficient saline, glucose, or other tonicity agent. These particular aqueous solutions are especially suitable for intravenous, intramuscular, subcutaneous, or intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected into the proposed site of infusion (see, e.g., “Remington's Pharmaceutical Sciences” (15^{th} ed.), pp. 1035-1038, 1570-1580). Some variation in dosage will necessarily occur depending on the condition of the subject being treated. The person responsible for administration will, in any event, determine the appropriate dose for the individual subject. Compounds and compositions according to the invention can also be formulated for parenteral administration by bolus injection or continuous infusion and can be presented in unit dose form, for instance as ampules, vials, small volume infusions, or pre-filled syringes, or in multi-dose containers with an added preservative.

Another route of administration of compositions according to the present invention is nasally, using dosage forms such as nasal solutions, nasal sprays, aerosols, or inhalants. Nasal solutions are usually aqueous solutions designed to be administered to the nasal passages in drops or sprays. Nasal solutions are typically prepared so that they are similar in many respects to nasal secretions, so that normal ciliary action is maintained. Thus, the aqueous nasal solutions usually are isotonic and/or slightly buffered in order to maintain a pH of from about 5.5 to about 6.5. In addition, antimicrobial preservatives, similar to those used in ophthalmic preparations, and/or appropriate drug stabilizers, if required, can be included in the formulation. Various commercial nasal preparations are known and can include, for example, antibiotics or antihistamines. Spray compositions can be formulated, for example, as aqueous solutions or suspensions or as aerosols delivered from pressurized packs, with the use of a suitable propellant, such as dichlorodifluoromethane, trichlorofluoromethane, dichlorotetrafluoroethane, 1,1,1,2,3,3,3-heptafluoropropane, 1,1,1,2-tetrafluoroethane, carbon dioxide, or other suitable gas.

Additional formulations that are suitable for other modes of administration include vaginal suppositories and/or pessaries. A rectal pessary or suppository can also be used. Suppositories are solid dosage forms of various weights or shapes, usually medicated, for insertion into the rectum, vagina, or urethra. After insertion, suppositories soften, melt, and/or dissolve into the cavity fluids. In general, for suppositories, traditional binders or carriers can include polyalkylene glycols, cocoa butter, or triglycerides.

Other dosage forms, including but not limited to ointments, creams, lotions, powders, or creams, can alternatively be used. Ointments and creams can, for example, be formulated with an aqueous or oily base with the addition of suitable gelling agents and/or solvents. Such bases, can thus, for example, include water and/or an oil such as liquid paraffin or a vegetable oil such as arachis, (peanut) oil or castor oil or a solvent such as a polyethylene glycol. Thickening agents which can be used include soft paraffin, aluminum stearate, cetostearyl alcohol, polyethylene glycols, microcrystalline wax, and beeswax. Lotions can be formulated with an aqueous or oily base and will in general also contain one or emulsifying agents, stabilizing agents, dispersing agents, suspending agents, or thickening agents.

Powders for external application can be formed with the aid of any suitable powder base, for example, talc, lactose, or starch.

Various factors must be taken into account in setting suitable dosages for active ingredients in pharmaceutical compositions according to the present invention. These factors include whether the patient is taking other medications that can alter the pharmacokinetics of the active ingredients, either causing them to be degraded or eliminated more rapidly or more slowly.

Toxicity and therapeutic efficacy of active ingredients in compositions according to the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

For any composition used in the method of the invention, the therapeutically effective dose can be estimated initially from cell culture assays. For example, a dose can be formulated in animal models to achieve a circulating plasma concentration range that includes the IC₅₀ as determined in cell culture (i.e., the concentration of the test compound which achieves a half-maximal improvement in a measurable clinical parameter when chronic effects are considered). Such information can be used to more accurately determine useful doses in humans. Levels in plasma may be measured, for example, by HPLC.

The exact formulation, route of administration and dosage can be chosen by the individual physician in view of the patient's condition. (See e.g. Fingl et al., in The Pharmacological Basis of Therapeutics, 1975, Ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administered dose in the management of the disorder of interest will vary with the severity of the condition to be treated and to the route of administration. The severity of the condition may, for example, be evaluated, in part, by standard prognostic evaluation methods. Further, the dose and perhaps the dose frequency, will also vary according to the age, body weight, and response of the individual patient. A program comparable to that discussed above may be used in veterinary medicine.

Depending on the specific conditions being treated, such agents may be formulated and administered systemically or locally. Typically, administration is systemic. Techniques for formulation and administration may be found in Remington's Pharmaceutical Sciences, 18th ed., Mack Publishing Co., Easton, Pa. (1990). Suitable routes may include oral, rectal, transdermal, vaginal, transmucosal, or intestinal administration; administration by inhalation; parenteral delivery, including intramuscular, subcutaneous, intramedullary injections, as well as intrathecal, direct intraventricular, intravenous, intraperitoneal, intranasal, or intraocular injections, just to name a few. Typically, oral administration is preferred.

For injection, compositions according to the present invention can be formulated in aqueous solutions. For transmucosal administration, penetrants appropriate to the barrier to be permeated are used in the formulation. Such penetrants are generally known in the art.

Use of pharmaceutically acceptable carriers to formulate pharmaceutical compositions according to the present invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

The invention is further described by the following Example. This Example is for illustrative purposes only and is not intended to limit the invention.

### EXAMPLE

### Effect of Bladderwrack Extract, Fucoidan, and Calcium D-Glucarate on Expression of mRNA for Cox-I, Cox-II, PGDH. Aromatase, and SF-1 (Prospective Example)

Endometrial biopsies collected from premenopausal women (<45 years of age with informed consent) undergoing benign gynecological hysterectomy at our institute will be used in these experiments. Samples from approximately 30 women will be collected to minimize the influence of potentially confounding factors such as age, duration of endometriosis, prior medical treatments for endometriosis, smoking, and menstrual cycle status. To minimize the effect of cycle stage samples will be collected from women on cycle days 18-24. The effects of treatments on (a) COX-I, COX-II, PGDH, P450_{AROM} and SF-I expression, (b) aromatase activity and (c) PGE₂ output will be determined in cell cultures of human eutopic and ectopic endometrium. Endometrial stromal cell cultures are routinely generated in my laboratory by established protocols. Briefly, endometrial cells will be dispersed and stromal cells collected via selective filtration and Ficoll purification. Cells will then be plated and treated with increasing log concentrations of the test compound in either 24 well plates (activity assays, PGE₂ measurements) or 100 mm Petri dishes (mRNA and protein expression studies). For protein determination, protein will be extracted from the cells using a commercially available reagent (Pierce, Rockford Ill.) and analyzed by quantitative Westerns for treatment effects on COX-I, COX-II, PGDH, aromatase and SF-I protein expression. Detection will be made using ECL. Band intensity will be quantified using imaging software, and protein expression between endometrium from women with and without endometriosis will be compared by t-test (α=0.05). To determine treatment effects of Fucoidan, bladderwrack extract, glucaric acid (calcium-d-glucarate) on mRNA expression, RNA will be extracted from the cells and the mRNA for COX-I, COX-II, PGDH, aromatase and SF-I will be quantified by RT-PCR. Aromatase activity will be determined by measuring the formation of ³H₂O by endometrial stromal cells cultured with 1β-³H androstenedione. Total COX activity will be determined using a commercially available assay, and prostaglandin output will be determined by a commercially available EIA kit for PGE₂ and PGF_{2α}. To confirm the purity of the stromal cell preparation cells from each culture will be stained immunohistochemically with cytokeratin and vimentin.

### ADVANTAGES OF THE INVENTION

Compositions and methods according to the present invention provide an improved way of treating a number of chronic, hard-to-treat conditions related to the effects of estrogen, particularly endometriosis. They can be used together with other therapies for symptoms such as pain, if desired, do not cause significant side effects, and are well tolerated.

Moreover, the use of compositions and methods according to the present invention provide rapid relief for these chronic conditions, especially endometriosis. This is particularly important and provides a clear advantage over previous treatment methods for endometriosis. For example, they do not pose the risk of side effects that treatment with gonadotropin-releasing hormone antagonists or danazol poses, such as increased cholesterol levels, insomnia, disturbances of sexual functioning, or depression. They also directly treat the hormonal basis of the condition and do not offer merely palliative and symptomatic relief, as do the use of pain-relieving drugs. Additionally, they can be used for long periods of time, which is very important for a chronic condition whose symptoms and effects typically last for years.

Additionally, compositions and methods according to the present invention exert an anti-inflammatory effect and an anti-adhesive effect. The latter effect inhibits the activity of cellular adhesion molecules. These effects contribute to a substantial reduction in inflammation and pain. The inhibition of the activity of cellular adhesion molecules prevents the progression of endometriosis, which is mediated by cells migrating out of the uterus and adhering to new surfaces.

Compositions and methods according to the present invention provide an effective treatment for endometriosis and other conditions associated with estrogen and do so by using components not previously suggested, in combination, for these conditions. This suggests the existence of a synergistic effect not previously known or appreciated.

Compositions and methods according to the present invention also inhibit the expression of at least one protein selected from the group consisting of aromatase, SF-I, COX-I, COX-II, and 15-hydroxyprostaglandin dehydrogenase at the level of transcription. This provides a new molecular mechanism for regulating prostaglandin synthesis and activity and preventing overproduction of these molecules.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

I claim:

1. A pharmaceutical composition for the treatment of an estrogen-dependent disease or condition, the composition comprising:
(a) at least one polysaccharide selected from the group consisting of an alginate and a fucoidan in a quantity effective to treat an estrogen-dependent disease or condition; and
(b) a pharmaceutically acceptable carrier.

2. The composition of claim 1 wherein the at least one polysaccharide comprises an alginate and a fucoidan.

3. The composition of claim 2 wherein the fucoidan is selected from the group consisting of U-fucoidan and F-fucoidan.

4. The composition of claim 2 wherein the fucoidan is isolated from Fucus vesiculosus.

5. The composition of claim 2 wherein the alginate is a polymer of guluronic acid and mannuronic acid.

6. The composition of claim 1 wherein the at least one polysaccharide comprises an alginate.

7. The composition of claim 6 wherein the alginate is a polymer of guluronic acid and mannuronic acid.

8. The composition of claim 1 wherein the composition comprises a fucoidan.

9. The composition of claim 8 wherein the fucoidan is isolated from Fucus vesiculosus.

10. The composition of claim 1 wherein the at least one polysaccharide is modified by a reaction selected from the group consisting of additional sulfation and methylation.

11. The composition of claim 1 wherein the at least one polysaccharide is obtained by bacterial fermentation.

12. The composition of claim 1 wherein the at least one polysaccharide is obtained from an animal source.

13. The composition of claim 1 wherein the composition comprises a quantity of polysaccharide such that the daily dose of the polysaccharide that is administered is from about 10 mg to about 3000 mg.

14. The composition of claim 14 wherein the composition comprises a quantity of polysaccharide such that the daily dose of the polysaccharide that is administered is from about 100 mg to about 1500 mg.

15. The composition of claim 1 further including a polymer of glucaric acid in a quantity sufficient to inhibit the enzyme β-glucuronidase.

16. The composition of claim 15 wherein the polymer of glucaric acid is calcium D-glucarate.

17. The composition of claim 16 wherein the quantity of calcium D-glucarate is such that the daily dose of the calcium D-glucarate is from about 50 mg to about 5 g.

18. The composition of claim 17 wherein the quantity of calcium D-glucarate is such that the daily dose of the calcium D-glucarate is from about 100 mg to about 3000 mg.

19. The composition of claim 1 further including D-glucaro-1,4-lactone in a quantity sufficient to inhibit the enzyme β-glucuronidase.

20. The composition of claim 1 further including at least one compound selected from the group consisting of diindolylmethane and indole-3-carbinol in a quantity sufficient to inhibit the activity of estrogen.

21. The composition of claim 1 wherein the composition further includes an aromatase inhibitor in a quantity sufficient to inhibit aromatase.

22. The composition of claim 21 wherein the aromatase inhibitor is selected from the group consisting of anastrozole, formestane, exemestane, vorozole, and letrozole.

23. The composition of claim 1 wherein the composition further includes progesterone or a progestin in a quantity sufficient to inhibit the effects of estrogen.

24. The composition of claim 1 wherein the composition further includes a lignan from an algal source in a quantity sufficient to act as an antagonist for the binding of estrogen to estrogen receptors.

25. The composition of claim 1 wherein the composition further includes a sterol from an algal source in a quantity sufficient to act as an antagonist for the binding of estrogen to estrogen receptors.

26. The composition of claim 1 wherein the estrogen-dependent disease or condition is endometriosis.

27. The composition of claim 1 wherein the composition has the activity of inhibiting the expression of at least one protein selected from the group consisting of aromatase, SF-I, COX-I, COX-II, and 15-hydroxyprostaglandin dehydrogenase at the level of transcription.

28. A pharmaceutical composition for the treatment of an estrogen-dependent disease or condition, the composition comprising:
(a) calcium D-glucarate in a quantity sufficient to treat an estrogen-dependent condition;
(b) diindolylmethane in a quantity sufficient to treat an estrogen-dependent condition; and
(c) a pharmaceutically acceptable carrier.

29. The composition of claim 28 wherein the composition comprises a quantity of calcium D-glucarate such that the daily dose of calcium D-glucarate is from about 100 mg to about 3000 mg, and the composition comprises a quantity of diindolylmethane such that the daily dose of diindolylmethane is from about 100 mg to about 3000 mg.

30. The composition of claim 28 wherein the composition further includes an aromatase inhibitor in a quantity sufficient to inhibit aromatase.

31. The composition of claim 28 wherein the composition further includes progesterone or a progestin in a quantity sufficient to inhibit the effects of estrogen.

32. The composition of claim 28 wherein the composition further includes a lignan from an algal source in a quantity sufficient to act as an antagonist for the binding of estrogen to estrogen receptors.

33. The composition of claim 28 wherein the composition further includes a sterol from an algal source in a quantity sufficient to act as an antagonist for the binding of estrogen to estrogen receptors.

34. The composition of claim 28 wherein the estrogen-dependent disease or condition is endometriosis.

35. The composition of claim 28 wherein the composition has the activity of inhibiting the expression of at least one protein selected from the group consisting of aromatase, SF-I, COX-I, COX-II, and 15-hydroxyprostaglandin dehydrogenase at the level of transcription.
